(19) United States
(12) Patent Application Publication
(10) Pub. No.: US 2022/0030860 A1
(43) Pub. Date: Feb. 3, 2022

(54) THERAPEUTIC COMPOSITION AND RELATED METHODS

(71) Applicant: 3M INNOVATIVE PROPERTIES COMPANY, St. Paul, MN (US)

(72) Inventors: Petra L. Kohler Riedi, Minneapolis, MN (US); Jimmie R. Baran, Jr., Prescott, WI (US); Jie Yang, Woodbury, MN (US); Ranjani V. Parthasarathy, Woodbury, MN (US)

(21) Appl. No.: 17/263,663

(22) PCT Filed: Aug. 5, 2019
(86) PCT No.: PCT/IB2019/056648

§ 371 Date: Jan. 27, 2021

Related U.S. Application Data
(60) Provisional application No. 62/715,991, filed on Aug. 8, 2018.

(51) Int. Cl. A01N 37/20; A01N 47/44; A01P 1/00; A61K 8/43; A61K 8/42; A61Q 17/00

(52) U.S. Cl. CPC A01N 37/20; A01N 47/44; A61Q 17/005; A61K 8/43; A61K 8/42; A01P 1/00

(57) ABSTRACT

A composition. The composition includes a compound of Formula (I) HOCH₂—(═CHOH═)ₙ—CH2NR¹R² (I) wherein R¹ and R² are independently selected from the group consisting of a hydrogen atom, an alkyl group, C(O)R³, and SO₂R⁴, wherein at least one of R¹ and R² is C(O)R³ or SO₂R⁴, R³ and R⁴ are selected from the group consisting of an alkyl group having seven carbon atoms, an alkyl group having eight carbon atoms, and an alkyl group having nine carbon atoms, and n is an integer from about 2 to about wherein a concentration of the compound of Formula (I) is more than its critical micelle concentration; and an antimicrobial agent selected from the group consisting of a biguanide antimicrobial agent and a polymeric, cationic, non-micelle forming antimicrobial material; wherein a concentration of the antimicrobial agent is no more than 5 wt %.

### BACKGROUND

The ability to fight bacteria has been a longstanding and valued feature in medical care products. To date, these benefits have been attained via antimicrobial agents. The use of antimicrobial agents such as antibiotics plays an important part in current medical therapy. For decades medicine has relied primarily upon antibiotics to fight systemic as well as topical infections. However, due to safety and environmental concerns with antimicrobial agents, there is still a need for a better antimicrobial compositions.

### SUMMARY

Thus, in one aspect, the present disclosure provides a composition, comprising: a compound of Formula (I) HOCH₂—(═CHOH═)ₙ—CH2NR¹R² (I) wherein R¹ and R² are independently selected from the group consisting of a hydrogen atom, an alkyl group, and C(O)R³, wherein at least one of R¹ and R² is C(O)R³, R³ is selected from the group consisting of an alkyl group having seven carbon atoms, an alkyl group having eight carbon atoms, and an alkyl group having nine carbon atoms, and n is an integer from about 2 to about 5; wherein a concentration of the compound of Formula (I) is more than its critical micelle concentration; and an antimicrobial agent selected from the group consisting of a biguanide antimicrobial agent and a polymeric, cationic, non-micelle forming antimicrobial material; wherein a concentration of the antimicrobial agent is no more than 5 wt %.

In another aspect, the present disclosure provides a composition, comprising: a compound of Formula (I) HOCH₂—(═CHOH═)ₙ—CH2NR¹R² (I) wherein R¹ and R² are independently selected from the group consisting of a hydrogen atom, an alkyl group, and C(O)R³, wherein at least one of R¹ and R² is C(O)R³, R³ is selected from the group consisting of an alkyl group having seven carbon atoms, an alkyl group having eight carbon atoms, and an alkyl group having nine carbon atoms, and n is an integer from about 2 to about 5; wherein a concentration of the compound of Formula (I) is more than 0.2 wt %; and an antimicrobial agent selected from the group consisting of a biguanide antimicrobial agent and a polymeric, cationic, non-micelle forming antimicrobial material; wherein a concentration of the antimicrobial agent is no more than 5 wt %.

In another aspect, the present disclosure provides a method, comprising: providing the composition of the present disclosure; and applying the composition to a surface of a subject or a medical device.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Drawings and the Detailed Description that follow more particularly exemplify certain embodiments using the principles disclosed herein.

### DEFINITIONS

For the following defined terms, these definitions shall be applied for the entire Specification, including the claims, unless a different definition is provided in the claims or elsewhere in the Specification based upon a specific reference to a modification of a term used in the following definitions:

The terms “about” or “approximately” with reference to a numerical value or a shape means +/−five percent of the numerical value or property or characteristic, but also expressly includes any narrow range within the +/−five percent of the numerical value or property or characteristic as well as the exact numerical value. For example, a temperature of “about” 100° C. refers to a temperature from 95° C. to 105° C., but also expressly includes any narrower range of temperature or even a single temperature within that range, including, for example, a temperature of exactly 100° C. For example, a viscosity of “about” 1 Pa-sec refers to a viscosity from 0.95 to 1.05 Pa-sec, but also expressly includes a viscosity of exactly 1 Pa-sec. Similarly, a perimeter that is “substantially square” is intended to describe a geometric shape having four lateral edges in which each lateral edge has a length which is from 95% to 105% of the length of any other lateral edge, but which also includes a geometric shape in which each lateral edge has exactly the same length.

The term “substantially” with reference to a property or characteristic means that the property or characteristic is exhibited to a greater extent than the opposite of that property or characteristic is exhibited. For example, a substrate that is “substantially” transparent refers to a substrate that transmits more radiation (e.g. visible light) than it fails to transmit (e.g. absorbs and reflects). Thus, a substrate that transmits more than 50% of the visible light incident upon its surface is substantially transparent, but a substrate that transmits 50% or less of the visible light incident upon its surface is not substantially transparent.

The term “CMC (critical micelle concentration)” with reference to a concentration of a surfactant in a liquid (eg water) is when the air/liquid interface is completely saturated with values of n. In these embodiments, the average value of n of a composition may be a non-integer.

In some embodiments, the compound of Formula (I) can be MEGA-8, MEGA-9, or MEGA-10. For example, when the compound of Formula (I) is MEGA-8, n is 4, R¹ is CH₃ and R² is C(O)R³, where R³ is C₈H₁₇.

In some embodiments, the composition comprises an antimicrobial agent selected from the group consisting of a biguanide antimicrobial agent and a polymeric, cationic, non-micelle forming antimicrobial material.

The biguanide antimicrobial agent can include those described in US20160213001 (Parthasarathy and Scholz et al). In some embodiments, the biguanide antimicrobial agent can include chlorhexidine (which is the common name for the antiseptic 1, 1′-hexamethylenebis-[5-(4-chlorophenyl)-biguanide] widely used in the form of its salts (such as the acetate, hydrochloride, and gluconate salts) in the cosmetic and pharmaceutical fields and also in cleaning preparations). Other salts of chlorhexidine include formate, lactate, isethionate, succinamate, glutamate, mono-diglycollate, dimethanesulfonate, di-isobutyrate, glucoheptonate. Preferably, the chlorhexidine salts are gluconate and acetate, the most preferred being chlorhexidine digluconate.

Additional examples of antimicrobial biguanide agents, which can be utilized in the present invention can include N¹-(4 chlorobenzyl)-N⁵-(2,4-dichlorobenzyl)-biguanide; p-chlorophenyl biguanide; 4-chlorobenzhydryl biguanide; N-3-lauroxypropyl-N⁵-p-chlorobenzyl biguanide; chlorophenyl-N⁵-lauryl biguanide; Olanedine (olanexidine gluconate) and the non-toxic addition salts thereof, especially gluconates and acetates. Polymeric biguanide antimicrobial agents such as polyhexamethylene biguanide and its salts can also be used.

The polymeric, cationic, non-micelle forming antimicrobial material can include those described in U.S. Pat. No. 8,338,491 (Asmus and Hobbs). In some embodiments, the polymeric, cationic, non-micelle forming antimicrobial material can include polymers having quaternary amine groups. These polymers typically have at least one alkyl or aralkyl chain of at least 6 carbon atoms and preferably at least 8 carbon atoms. The polymers may be linar, branched, hyperbranched, or dendrimers. Polymeric, cationic, non-micelle forming antimicrobial materials can include polyethyleneimine (PEI), polymeric quaternary ammonium salts, or chitosan.

In some embodiments, the composition can further comprise a glucamine compound. Glucamine compound can be any suitable glucamine compound, for example, amino sugar alcohols and derivatives, including D-glucamine, N-methyl-D-glucamine, N-Ethyl-D-Glucamine, N-Propyl-Glucamine, N-Butyl-D-Glucamine, N-octyl-D-glucamine, and combinations thereof.

In some embodiments, the composition can further comprise a glucosamine compound. Glucosamine compound can be any suitable glucamine compound, for example, D-Glucosamine Hydrochloride, D-Glucosamine Sulfate, D-Glucosamine Phosphate, D-Glucosamine gluconate and combinations thereof.

In some embodiments, the composition can further comprise a pharmaceutically acceptable carrier or solvent. The pharmaceutically acceptable carrier may comprise a liquid, a solid, or a gel. In some embodiments, the carrier may be a liquid at about room temperature. In other embodiments, the carrier may be a solid at about room temperature. In some embodiments, the carrier may be a liquid at about the temperature of the oral cavity of a human, i.e., at about 37° C. In other embodiments, the carrier may be a solid at about the temperature of the oral cavity of a human. Exemplary liquid carriers include water, alcohol, glycerol, polyethylene glycol, triethanolamine, methoxypropanol, isopropanol, ethyl acetate, polypropylene glycol and a combination thereof. Exemplary solid carriers include polymers such as natural rubber, butyl rubber, poly(isobutylene), elastomers, styrene-butadiene rubber, polysaccharides, and waxes (e.g., beeswax).

Each non-carrier component of the composition may independently be dissolved, dispersed, suspended, or emulsified in the carrier. In some embodiments, at least one component of the composition is dissolved in the carrier. In some embodiments, at least one component of the composition is dispersed in the carrier. In some embodiments, at least one component of the composition is suspended in the carrier. In some embodiments, at least one component of the composition is emulsified in the carrier.

In some embodiments, the composition can further comprise a binder. The binder may provide a reservoir of a composition comprising a compound of Formula I. The composition may be released from the binder. The binder may hold a composition comprising a compound of Formula I. In some embodiments, the binder can comprise an acrylic polymer. Suitable acrylic polymers include polymers and copolymers of lower alkyl esters of acrylic or methacrylic acids. In some embodiments, the binder can comprise natural polysaccharides and derivatives selected from murein, pectins, hyaluronate, chondroitin-4-sulfate, dermatan sulfate, keratan sulfate, heparin, heparan sulfate, hydroxyethyl cellulose, hydroxypropyl cellulose, guar gum, and combinations thereof.

In some embodiments, the composition can further comprise sugar-alcohol humectant including xylitol, sorbitol, mannitol and erythritol. In some embodiments, the composition can be in a form selected from the group consisting of a solution, a dispersion, a suspension, an emulsion, a solid, a paste, a foam, a gel, a spray, a wipe, a mitt and a brush. Any component of the composition may be dissolved, dispersed, suspended, or emulsified in any other component of the composition. In some embodiments, the components are mutually soluble (i.e., miscible with each other).

In some embodiments, the composition may comprise a concentration of the compound of Formula (I) more than its critical micelle concentration (CMC), more than 120% of its CMC, more than 130% of its CMC, more than 140% of its CMC, more than 150% of its CMC, more than 160% of its CMC, more than 170% of its CMC, more than 180% of its CMC, more than 190% of its CMC, more than 200% of its CMC, more than 250% of its CMC, more than 300% of its CMC, more than 400% of its CMC, or more than 500% of its CMC. CMC used in the current disclosure refers to the CMC of the compound of Formula (I) alone in water at 25° C.

In some embodiments, the composition may comprise a concentration of the compound of Formula (I) more than 0.2 wt %, more than 0.6 wt %, more than 1.8 wt %, more than 1.8 wt %, more than 2 wt %, more than 4 wt %, more than 6 wt %, more than 10 wt %, more than 12 wt %, more than 16 wt %, more than 20 wt %, more than 30 wt %, more than 40 wt %

In certain embodiments, the composition may provide a concentration of a compound of Formula I, up to about the solubility limit of the compound, in a medium such as, for example, water, culture broth, or saliva. It is recognized that the solubility limit may be different in different media. In other embodiments, the composition may comprise a concentration of a compound of Formula I, greater than about the solubility limit of the compound, in a binder or in a pharmaceutically acceptable carrier.

In some embodiments, the composition may comprise a concentration of the antimicrobial agent no more than 5 wt %, no more than 4 wt %, no more than 3 wt %, or no more than 2 wt %. The composition of the current disclosure can provide a synergistic enhancement of antimicrobial activity for biguanide antimicrobial agents and polymeric, cationic, non-micelle forming antimicrobial materials, therefore allowing for the use of lower concentrations of antimicrobial materials in order to achieve microbial kill.

In another aspect, the invention provides a method of inhibiting microbial growth, comprising providing the composition of the current disclosure and applying the composition to a surface of a subject or a medical device. The surface can be a surface in the oral cavity of a subject includes, for example, a buccal surface, a gingival surface, a tooth, a dental restoration, and bone. The composition may be applied to the oral cavity of a subject by, for example, biguanide antimicrobial agent and a polymeric, cationic, non-micelle forming antimicrobial material; wherein a concentration of the antimicrobial agent is no more than 5 wt %.

Embodiment 6 is the composition of embodiment 5, wherein the concentration of the compound of Formula (I) is more than 0.6 wt %.
Embodiment 7 is the composition of embodiment 5, wherein the concentration of the compound of Formula (I) is more than 1.8 wt %.
Embodiment 8 is the composition of any one of embodiments 1-7, wherein the concentration of the antimicrobial agent is no more than 4 wt %.
Embodiment 9 is the composition of any one of embodiments 1-8, wherein the concentration of the antimicrobial agent is no more than 3 wt %.
Embodiment 10 is the composition of any one of embodiments 1-9, further comprising a glucamine compound.
Embodiment 11 is the composition of embodiment 10, wherein the glucamine compound is selected from the group of D-glucamine, N-methyl-D-glucamine, N-Ethyl-D-Glucamine, N-Propyl-Glucamine, N-Butyl-D-Glucamine, N-octyl-D-ghicamine, and combinations thereof.
Embodiment 12 is the composition of any one of embodiments 1-11, further comprising a glucosamine compound.
Embodiment 13 is the composition of embodiment 12, wherein the glucosamine compound is selected from the group of D-Glucosamine Hydrochloride, D-Glucosamine Sulfate, D-Glucosamine Phosphate, D-Glucosamine gluconate and combinations thereof.
Embodiment 14 is the composition of any one of embodiments 1-13, further comprising a pharmaceutically acceptable carrier or solvent selected from the group consisting of water, alcohol, glycerol, polyethylene glycol, triethanolamine, methoxypropanol, isopropanol, ethyl acetate, polypropylene glycol and a combination thereof.
Embodiment 15 is the composition of any one of embodiments 1-14, wherein the compound of Formula (I) is MEGA-8, MEGA-9, or MEGA-10.
Embodiment 16 is the composition of any one of embodiments 1-15, further comprising a binder.
Embodiment 17 is the composition of any one of embodiment 16, wherein the binder is natural polysaccharides and derivatives selected from murein, pectins, hyaluronate, chondroitin-4-sulfate, dermatan sulfate, keratan sulfate, heparin, heparan sulfate, hydroxyethyl cellulose, hydroxypropyl cellulose, guar gum, and combinations thereof.
Embodiment 18 is the composition of any one of embodiments 1-17, further comprising sugar-alcohol humectant including xylitol, sorbitol, mannitol and erythritol.
Embodiment 19 is the composition of any one of embodiments 1-18, wherein the composition is in a form selected from the group consisting of a solution, a dispersion, a suspension, an emulsion, a solid, a paste, a foam, a gel, a spray, a wipe, a mitt and a brush.
Embodiment 20 is a method, comprising:

providing the composition of any one of embodiments 1-19; and

applying the composition to a surface of a subject or a medical device.

Embodiment 21 is a composition, comprising:

nonanoyl-N-methylglucamide;

wherein the concentration of nonanoyl-N-methylglucamide is greater than 20 mM; and

an antimicrobial agent selected from the group consisting of a biguanide antimicrobial agent and a polymeric, cationic, non-micelle forming antimicrobial material;

wherein the concentration of the antimicrobial agent is no more than 5 wt %.

Embodiment 22 is the composition of embodiment 21, wherein the concentration of nonanoyl-N-methylglucamide is greater than or equal to 22 mM.
Embodiment 23 is the composition of embodiment 21, wherein the concentration of nonanoyl-N-methylglucamide is greater than or equal to 25 mM.

1. A composition, comprising:
a compound of Formula (I)
HOCH₂—(═CHOH═)ₙ—CH2NR¹R² (I)
wherein R¹ and R² are independently selected from the group consisting of a hydrogen atom, an alkyl group, C(O)R³, and SO₂R⁴;
wherein at least one of R¹ and R² is C(O)R³ or SO₂R⁴, R³ and R⁴ are selected from the group consisting of an alkyl group having seven carbon atoms, an alkyl group having eight carbon atoms, and an alkyl group having nine carbon atoms, and n is an integer from about 2 to about 5;
wherein a concentration of the compound of Formula (I) is more than its critical micelle concentration; and
an antimicrobial agent selected from the group consisting of a biguanide antimicrobial agent and a polymeric, cationic, non-micelle forming antimicrobial material;
wherein a concentration of the antimicrobial agent is no more than 5 wt %.

2. The composition of claim 1, wherein the concentration of the compound of Formula (I) is more than 120% of its critical micelle concentration .

3. The composition of claim 1, wherein the concentration of the compound of Formula (I) is more than 150% of its critical micelle concentration.

4. The composition of claim 1, wherein the concentration of the compound of Formula (I) is more than 180% of its critical micelle concentration.

5. A composition, comprising:
a compound of Formula (I)
HOCH₂—(═CHOH═)ₙ—CH2NR¹R² (I)
wherein R¹ and R² are independently selected from the group consisting of a hydrogen atom, an alkyl group, C(O)R³, and SO₂R⁴,
wherein at least one of R¹ and R² is C(O)R³ or SO₂R⁴, R³ and R⁴ are selected from the group consisting of an alkyl group having seven carbon atoms, an alkyl group having eight carbon atoms, and an alkyl group having nine carbon atoms, and n is an integer from about 2 to about 5;
wherein a concentration of the compound of Formula (I) is more than 0.2 wt %; and
an antimicrobial agent selected from the group consisting of a biguanide antimicrobial agent and a polymeric, cationic, non-micelle forming antimicrobial material;
wherein a concentration of the antimicrobial agent is no more than 5 wt %.

6. The composition of claim 5, wherein the concentration of the compound of Formula (I) is more than 0.6 wt %.

7. The composition of claim 5, wherein the concentration of the compound of Formula (I) is more than 1.8 wt %.

8. The composition of claim 1, wherein the concentration of the antimicrobial agent is no more than 4 wt %.

9. The composition of claim 1, wherein the concentration of the antimicrobial agent is no more than 3 wt %.

10. The composition of claim 1, further comprising a glucamine compound.

11. The composition of claim 10, wherein the glucamine compound is selected from the group of D-glucamine, N-methyl-D-glucamine, N-Ethyl-D-Glucamine, N-Propyl-Glucamine, N-Butyl-D-Glucamine, N-octyl-D-glucamine, and combinations thereof.

12. The composition of claim 1, further comprising a glucosamine compound.

13. The composition of claim 12, wherein the glucosamine compound is selected from the group of D-Glucosamine Hydrochloride, D-Glucosamine Sulfate, D-Glucosamine Phosphate, D-Glucosamine gluconate and combinations thereof.

14. The composition of claim 1, further comprising a pharmaceutically acceptable carrier or solvent selected from the group consisting of water, alcohol, glycerol, polyethylene glycol, triethanolamine, methoxypropanol, isopropanol, ethyl acetate, polypropylene glycol and a combination thereof.

15. The composition of claim 1, wherein the compound of Formula (I) is MEGA-8, MEGA-9, or MEGA-10.

16. The composition of claim 1, further comprising a binder.

17. The composition of any one of claim 16, wherein the binder is natural polysaccharides and derivatives selected from murein, pectins, hyaluronate, chondroitin-4-sulfate, dermatan sulfate, keratan sulfate, heparin, heparan sulfate, hydroxyethyl cellulose, hydroxypropyl cellulose, guar gum, and combinations thereof.

18. The composition of claim 1, further comprising sugar-alcohol humectant including xylitol, sorbitol, mannitol and erythritol.

19. The composition of claim 1, wherein the composition is in a form selected from the group consisting of a solution, a dispersion, a suspension, an emulsion, a solid, a paste, a foam, a gel, a spray, a wipe, a mitt and a brush.

20. A method, comprising:
providing the composition of claim 1; and
applying the composition to a surface of a subject or a medical device.
